(19) United States
(12) Patent Application Publication
(10) Pub. No.: US 2003/0050611 A1
(43) Pub. Date: Mar. 13, 2003

(54) Needle shield using external blunt shield member

(76) Inventor: Christopher Noel Cindrich, South Jordan, UT (US)

Correspondence Address: BECTON, DICKINSON AND COMPANY, 1 BECTON DRIVE, FRANKLIN LAKES, NJ, 07417-1880, US

(21) Appl. No.: 10/279,522

(22) Filed: Oct. 24, 2002

Related U.S. Application Data
(63) Continuation of application No. 09/731,961, filed on Dec. 7, 2000.

(51) Int. Cl. A61M 5/00

(52) U.S. Cl. 604/264

(57) ABSTRACT

An introducer needle assembly with a needle shield is provided. The needle shield includes a means for locking the needle shield in the shielded position.

This application is a continuation of earlier pending application Ser. No. 09/731,961 filed Dec. 7, 2000.

### BACKGROUND OF THE INVENTION

The subject invention relates to an introducer needle assembly that includes a needle shield that safely shields the sharp distal tip of the introducer needle after the introducer needle assembly has been used to insert a catheter or other medical device into a patient.

Catheters, particularly intravascular (IV) catheters, are used for infusing fluid, such as normal saline solution, various medicaments and total parenteral nutrition, into a patient, withdrawing blood from a patient or monitoring various parameters of the patient's vascular system. Peripheral IV catheters tend to be relatively short, and typically are on the order of about two inches or less in length. The most common type of IV catheter is an over-the-needle peripheral IV catheter. As its name implies, an over-the-needle catheter is mounted over an introducer needle having a sharp distal tip. At least the distal portion of the catheter tightly engages the outer surface of the needle to prevent peelback of the catheter and thus facilitates insertion of the catheter into the blood vessel. The catheter and the introducer needle are assembled so that the distal tip of the introducer needle extends beyond the distal tip of the catheter with the bevel of the needle facing up away from the patient's skin.

The catheter and introducer needle assembly is inserted at a shallow angle through the patient's skin into a blood vessel. There are many techniques for inserting such a catheter and introducer needle assembly into a patient. In one insertion technique, the introducer needle and catheter are inserted completely into the blood vessel together. In another technique, the introducer needle is partially withdrawn into the catheter after the initial insertion into the blood vessel. The catheter is then threaded over the needle and inserted completely into the blood vessel.

In order to verify proper placement of the catheter and introducer needle assembly in the blood vessel, the clinician confirms that there is flashback of blood in a flashback chamber. The flashback chamber is typically formed as part of the needle hub. Once proper placement of the catheter into the blood vessel is confirmed the clinician applies pressure to the blood vessel by pressing down on the patient's skin over the blood vessel distal of the introducer needle and the catheter. This finger pressure occludes or at least minimizes further blood flow through the introducer needle and the catheter. The clinician then withdraws the introducer needle, leaving the catheter in place, and attaches an appropriate device to the catheter. Such a device can include a fluid delivery device, a PRN, a deadender cap, or a blood pressure monitoring probe. Once the introducer needle is withdrawn from the catheter, the introducer needle is a “blood contaminated sharp” and must be properly handled.

Other catheters such as midline catheters, central catheters and peripherally inserted central catheters may also be used for certain types of IV therapy. Specifically these catheters are used when certain harsh medicaments must be infused into the patient and must be diluted quickly in a large vein to prevent adverse reaction by the patient. In such a situation, it is important for the distal tip of the catheter to be located in the auxiliary, subclavian or brachiocephalic vein or in the superior vena cava. Midline catheters, central catheters and peripherally inserted central catheters, which tend to be longer than standard perpheral IV catheters and can be on the order of several inches long or longer, fill this need.

For these longer catheters, guidewires can be used to facilitate the introduction and proper placement of the catheter into a patient. When a guidewire is used, an introducer can be placed in the patient's blood vessel in a manner similar to a standard venipuncture procedure for placing an IV catheter in the patient's blood vessel. Such an introducer can have a configuration similar to that of a standard peripheral IV catheter and can be disposed about an introducer needle assembly in much the same manner as a standard peripheral IV catheter is disposed about an introducer needle assembly. When the introducer is properly located in the patient and the introducer needle assembly removed, a guidewire is inserted into the introducer into the patient's vasculature. The guidewire is then maneuvered through the patient's vasculature until the distal tip of the guidewire is located in the proper position. Thereafter, the introducer is removed and the therapeutic catheter is inserted into the patient over the guidewire, which acts as a track to facilitate the proper placement of the therapeutic catheter in the patient. Alternatively, a separate introducer is not needed for the introduction of a guidewire into the patient. Instead, the introducer needle assembly itself may be used as the guidewire introducer. However, in both cases, once the introducer needle is withdrawn from the patient, the introducer needle is a “blood contaminated sharp” and must be properly handled.

In recent years, there has been great concern over the contamination of clinicians with a patient's blood and recognition that “blood contaminated sharps” must be disposed to avoid an accidental needle stick. This concern has arisen because of the advent of currently incurable and fatal diseases, such as Acquired Immunosuppressive Deficiency Syndrome (“AIDS”), which can be transmitted by the exchange of body fluids from an infected person to another person. Thus, contact with the body fluid of an AIDS infected person must be avoided. As noted above, if an introducer needle has been used to place a catheter or some other medical device in a blood vessel of an AIDS infected person, the introducer needle, via its sharp distal tip, is a vehicle for the transmission of the disease. Although clinicians are aware of the need to properly handle “blood contaminated sharps”, unfortunately in certain medical environments, such as emergency situations or as a result of inattention or neglect, needlesticks with a contaminated introducer needle still occur.

As a result of the problem of accidental needlesticks by “blood contaminated sharps”, various needle shields have been developed. Generally, such needle shields work for their intended purpose but could be improved. For example, some needle shields are bulky, difficult to use or require special features or techniques to be operative.

### SUMMARY OF THE INVENTION

It is therefore an object of this invention to provide a needle shield that is compact.

It is another object of this invention to provide a needle shield that is simple and easy to use.

It is still another object of this invention to provide a needle shield that requires no special features or techniques to be operative.

The needle shield of this invention includes a hollow blunt generally cylindrical needle shield into which the introducer needle extends. The needle shield has an external diameter only slightly larger than the external diameter of the introducer needle so the needle shield can be easily inserted into the patient along with the introducer needle. The needle shield is movable a short distance from a proximal position to a distal position. In the proximal position, the distal end of the shield is proximal of the sharp distal tip of the introducer needle. In the distal position, the distal end of the shield is distal of the sharp distal tip of the introducer needle. The shield is connected to a slide member that is disposed about the needle hub. Cooperating detent mechanisms on the slide member and the needle hub allow movement of the shield from the proximal position to the distal position and lock the shield in the distal position. The configuration of the detent mechanism makes the needle shield easy to use and difficult to defeat. The internal surface of the slide member adjacent to the proximal end of the shield has a generally funnel configuration that facilitates the introduction of another medical device, such as a guidewire into the introducer needle.

### BRIEF DESCRIPTION OF THE DRAWINGS

The preferred embodiments are illustrated in the drawings in which like reference numerals refer to like elements and in which:

FIG. 1 is a perspective view of the needle shield of this invention with the needle shield in the proximal position;

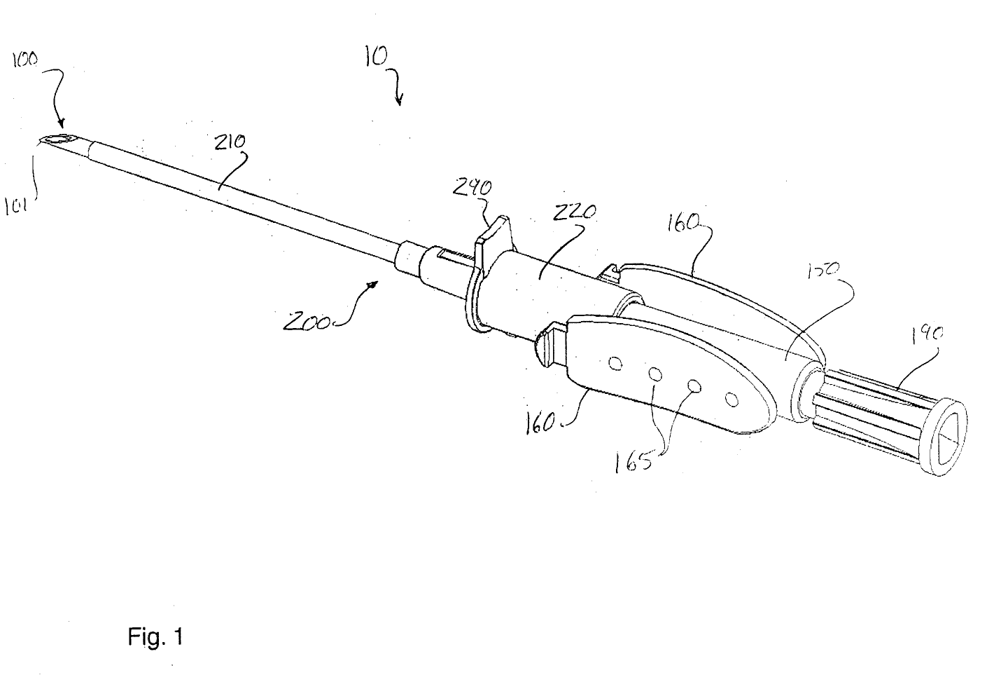

FIG. 2 is a perspective view of the needle shield of this invention with the needle shield in the distal, shielded position;

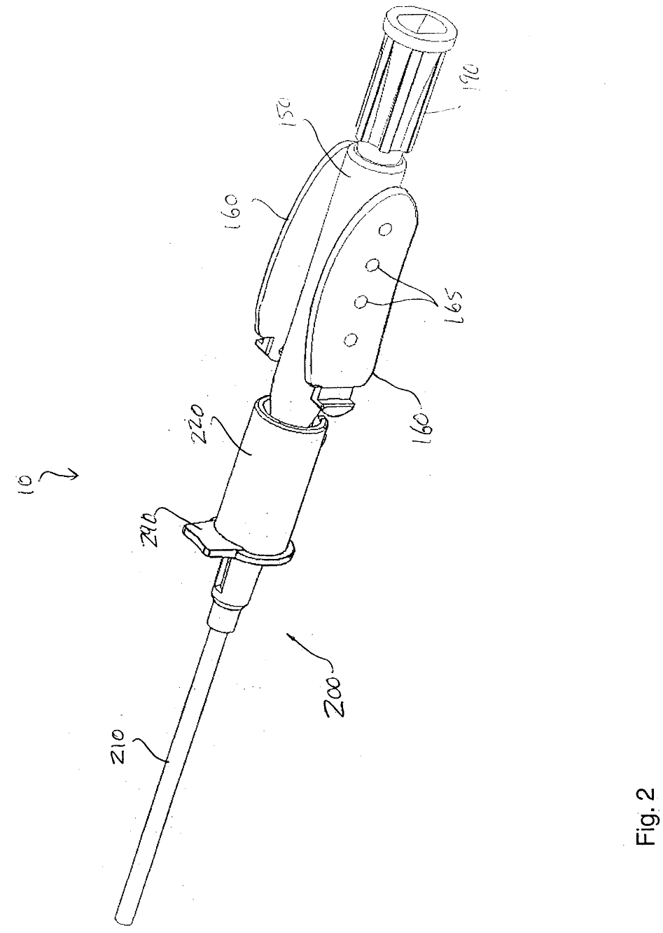

FIG. 3 is an exploded bottom perspective view of the needle shield of this invention and including a catheter associated with the needle shield;

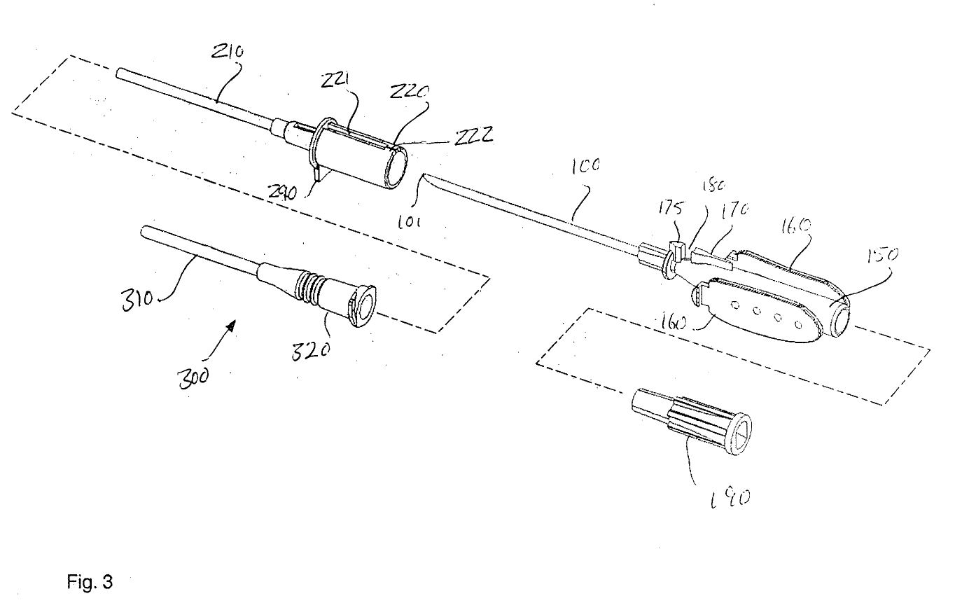

FIG. 4 is a side elevation cross sectional view of the needle shield of this invention with the needle shield in the proximal position and including a catheter associated with the needle shield;

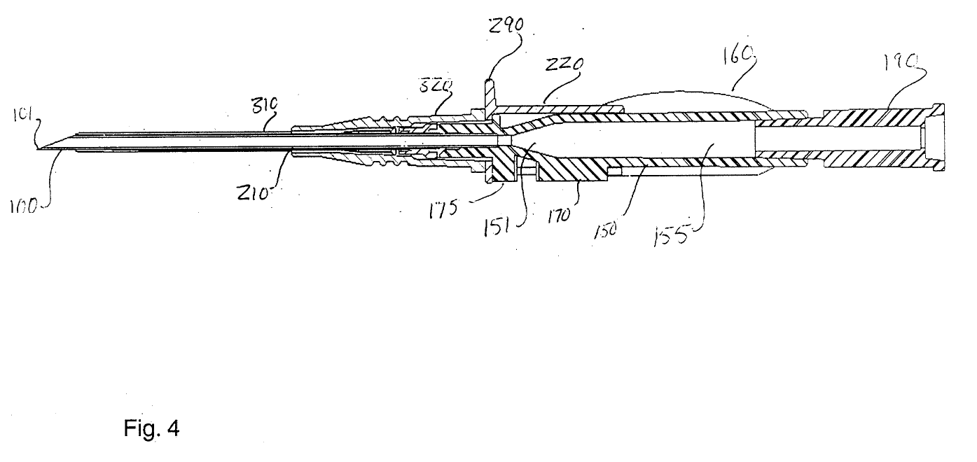

FIG. 5 is an enlarged view of a portion of the bottom of the needle shield of this invention with the needle shield in the proximal position and a catheter associated with the needle shield;

FIG. 6 is a side elevation cross sectional view of the needle shield of this invention with the needle shield in the distal, shielded position;

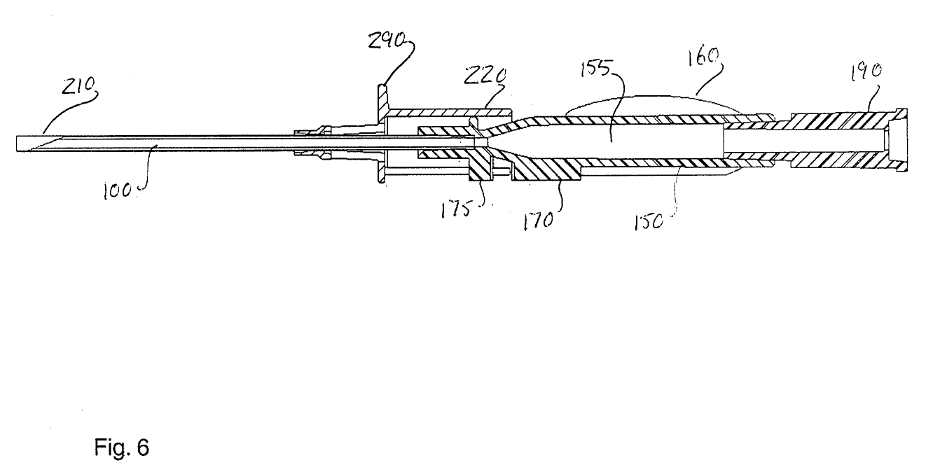

FIG. 7 is an enlarged view of a portion of the bottom of the needle shield of this invention with the needle shield in the distal, shielded position;

FIG. 8 is an enlarged perspective view in cross section of a portion of the needle shield of this invention with the needle shield in the proximal position including a catheter associated with the needle shield;

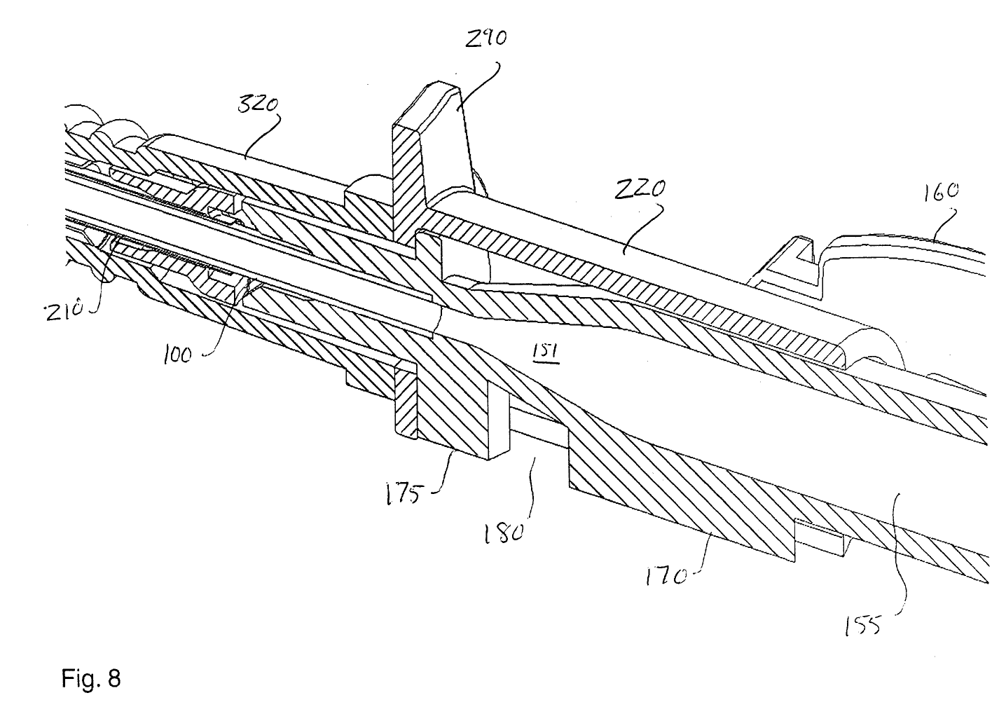

FIG. 9 is a top plan view of the needle shield of this invention with the needle shield in the proximal position and including a catheter associated with the needle shield;

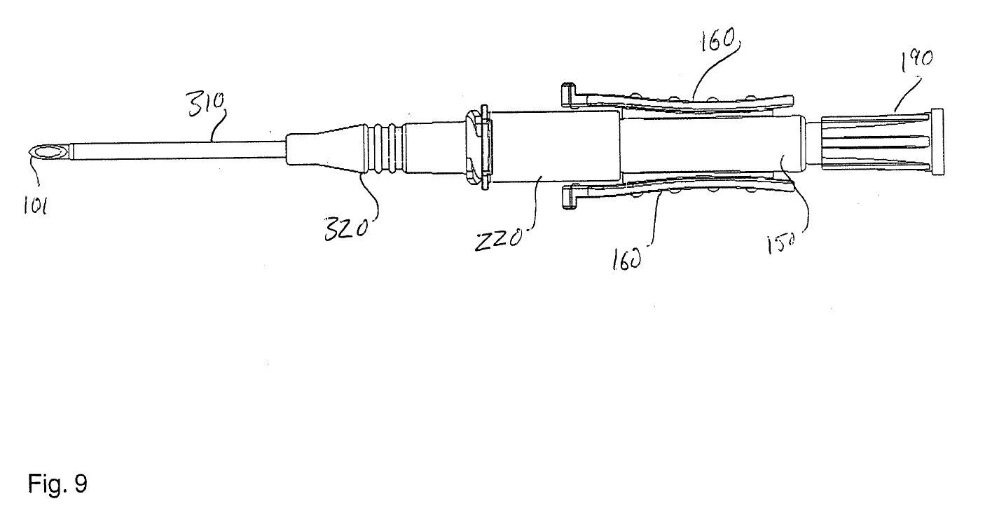

FIG. 10 is a bottom plan view of the needle shield of this invention with the needle shield in the proximal position and including a catheter associated with the needle shield; and

FIG. 11 is a side elevation view of the needle shield of this invention with the needle shield in the proximal position and including a catheter associated with the needle shield.

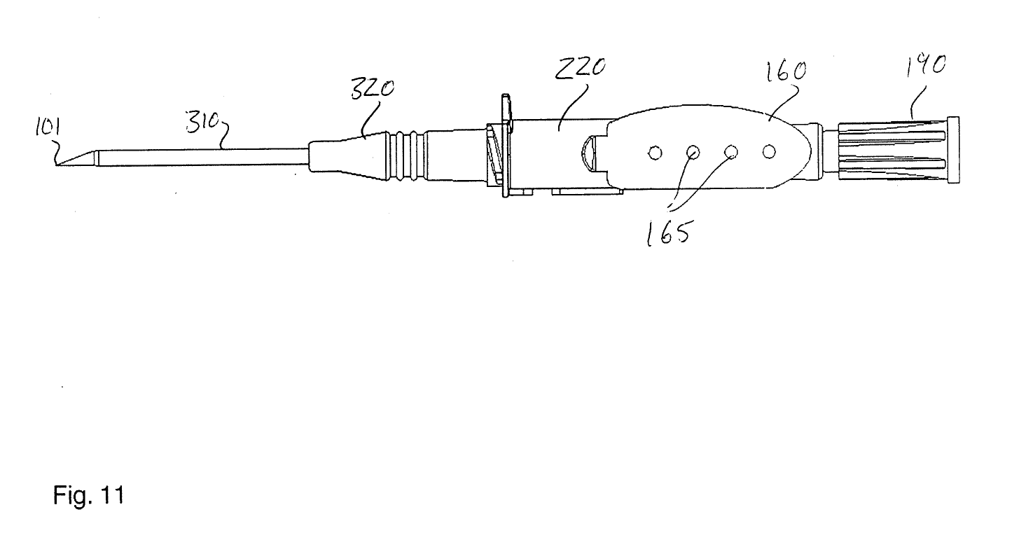

### DETAILED DESCRIPTION OF THE INVENTION

As used herein, the term “proximal” refers to a location on the needle shield of this invention that, during normal use, is closest to the clinician using the device and farthest from the patient in connection with whom the device is used. Conversely, the term “distal” refers to a location on the needle shield of this invention that, during normal use, is farthest from the clinician using the device and closest to the patient in connection with whom the device is used.

As used herein, the term “top”, “up” or “upwardly” refers to a location on the needle shield of this invention that, during normal use, is radially away from the longitudinal axis of the device and away from the patient's skin. Conversely, as used herein, the term “bottom”, “down” or “downwardly” refers to a location on the needle shield of this invention that, during normal use, is radially away from the longitudinal axis of the device and toward the patient's skin.

As used herein, the term “in” or “inwardly” refers to a location with respect to the needle shield of this invention that, during normal use, is toward the inside of the device. Conversely, as used herein, the term “out” or “outwardly” refers to a location with respect to the needle shield of this invention that, during normal use, is toward the outside of the device.

This invention is described herein using like reference numbers for like elements in the different embodiments. Although this invention is described herein in connection for use as an introducer needle assembly for a typical peripheral IV catheter as well as for a guidewire introducer, it is to be understood that this invention is applicable to other medical devices where it is desirable for a needle to be shielded after use. In addition, while this invention is satisfied by embodiments in many different forms, there are shown in the drawings and herein described in detail, preferred embodiments of the invention with the scope of the invention measured by the appended claims.

The introducer needle assembly 10 of this invention includes an introducer needle 100 and a needle shield 200. Introducer needle 100 has a sharp distal tip 101 defined by a bevel and a proximal end connected to the distal end of a needle hub 150. Introducer needle 100 is preferably formed from stainless steel. Materials that can be used to form needle hub 150 include, but are not limited to, thermoplastic polymeric resins such as polycarbonate, polystyrene, polypropylene and the like. Of course, if desired other materials could be used to form needle hub 150.

Needle hub 150 can include an integrated flashback chamber 155 defined by needle hub 150. Preferably, the distal end portion of the inside of needle hub 150 is formed with a funnel configuration 151 where the smaller diameter portion of funnel configuration 151 is connected to the proximal end of introducer needle 100. This configuration facilitates the insertion of another medical device, such as a guidewire, into the proximal end of introducer needle 100. In addition, the smaller portion of funnel configuration 151 enhances the initial visualization of blood when blood flows out of the proximal end of introducer needle 100 due to a faster blood flow front caused by the smaller portion of funnel configuration 151.

Since the proximal end of introducer needle 100 is connected to the distal end of needle hub 150, the resulting volume of integrated flashback chamber 155 defined by needle hub 150 is larger than typical introducer needle assemblies used for IV catheters or guidewire introducers. This larger volume provides better flashback visualization and enables a longer fill time to facilitate higher first stick success rates when using introducer needle assembly 10. Preferably, the proximal end of needle hub 150 is open. A vented plug 190 may be located in the open proximal end of needle hub 150 to provide a closed chamber inside needle hub 150. Vented plug 190 allows air but not fluid to flow therethrough. If desired, the proximal end of needle hub 150 can be formed with a luer type connection to facilitate the connection of another medical device, such as a syringe, to introducer needle assembly 10.

Needle hub 150 also includes a pair of finger grips 160 disposed on either side thereof. Each finger grip 160 has a generally elliptical configuration with a curved face and a plurality of raised touch bumps 165 disposed on its face. This configuration for finger grips 160 provides a stable and comfortable gripping surface for the clinician and a readily distinguishable finger placement location that keeps the clinician's fingers clear of flashback chamber 155.

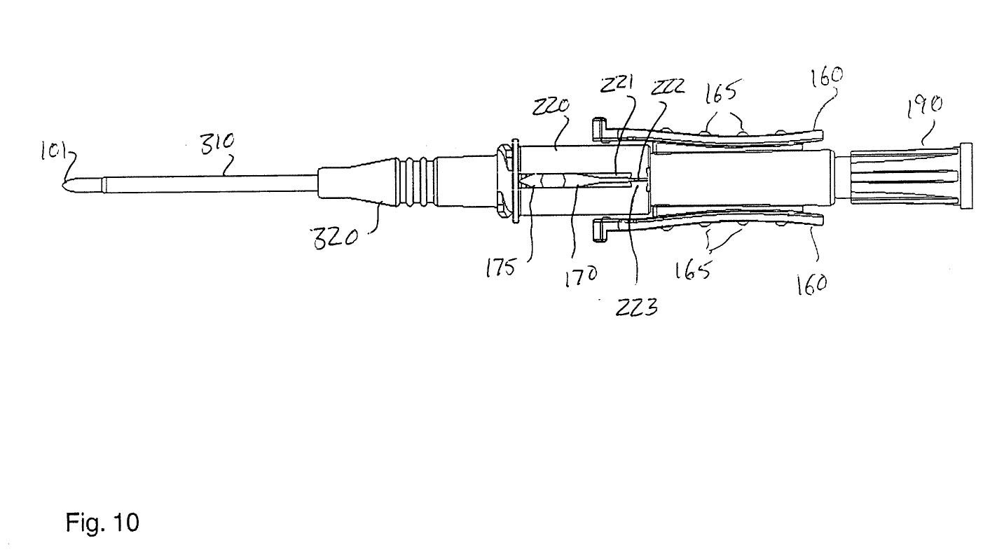

Needle hub 150 also includes a detent mechanism comprised of a proximal lock 170 and a distal lock 175. Preferably, proximal lock 170 and distal lock 175 are substantially longitudinally aligned and separated by a gap 180. When seen from a bottom plan view, e.g. FIG. 10, both proximal lock 170 and distal lock 175 have tapered sides that extend from a narrow portion to a wider portion. For proximal lock 170, the narrow portion faces proximally and the wider portion faces distally. For distal lock 175, the narrow portion faces distally and the wider portion faces proximally. Preferably, the distal end of proximal lock 170 includes a dogleg shaped surface 171, i.e. an angled surface, where the apex of the bend points proximally and is substantially aligned with the longitudinal axis of proximal lock 170. Preferably, the proximal end of distal lock 175 includes a dogleg shaped surface 176 where the apex of the bend points distally and is substantially aligned with the longitudinal axis of distal lock 175. As will be discussed below, the configuration of proximal lock 170 and distal lock 175 minimizes the force needed to activate needle shield 200, i.e. move needle shield 200 to the distal, shielded position, and maximizes the force needed to defeat needle shield 200, i.e. move needle shield 200 so as to reexpose sharp distal tip 101 of introducer needle 100.

Needle shield 200 includes an external blunt member 210 connected at its proximal end to the distal end of the blunt slide 220. Materials that can be used to form blunt slide 220 include, but are not limited to, thermoplastic polymeric resins such as polycarbonate, polystyrene, polypropylene and the like. External blunt member 210 is preferably formed from stainless steel. Of course, if desired other materials could be used to form blunt slide 220 and external blunt member 210.

External blunt member 210 is disposed about introducer needle 100 and blunt slide 220 is disposed about needle hub 150 so that needle shield 200 can move distally with respect to introducer needle 100 to shield sharp distal tip 101 of introducer needle 100 after use. Blunt slide 220 may include an upstanding tab 290 against which a clinician can push to facilitate distal movement of blunt slide 220 with respect to needle hub 150, which causes external blunt member 210 to move distally with respect to introducer needle 100. Needle shield 200 is designed to have as short a throw length as possible. The throw length is the distance of movement of blunt slide 220 with respect to needle hub 150 from the proximal position to the distal, shielded position for needle shield 200. The throw length should be short enough to make it easy for the clinician to use but long enough to allow external blunt member 210 to shield sharp distal tip 101 of introducer needle 100. A throw length of between about 0.1 inches and about 0.45 inches is desirable, with a throw length of about 0.38 inches preferred. This throw length is substantially shorter than the length covered by the movement of a typical clinician's finger from a clenched position to an extended position.

External blunt member 210 has an internal diameter slightly larger than the external diameter of introducer needle 100 to allow external blunt member 210 to easily move along introducer needle 100. The external diameter of external blunt member 210 preferably is only slightly larger than the external diameter of introducer needle 100. This provides for a compact needle shielding device and allows external blunt member 210 to fit inside catheter 310 and be easily inserted into the patient along with introducer needle 100.

Blunt slide 220 defines a slot 221 along a portion thereof and a slit 222 extending from slot 221 to the proximal end of blunt slide 220. Slot 221 and slit 222 define a pair of ears 223 on blunt slide 220. The length of slit 222 and thus the length of ears 223 is substantially equal to the length of gap 180 between proximal lock 170 and distal lock 175. The length of ears 223 should be chosen to maximize the shear strength of ears 223 while minimizing the throw length of blunt slide 220. Preferably, ears 223 should have a maximum length of between about 0.020 inches and about 0.150 inches, with a maximum length of about 0.060 inches preferred. In addition, ears 223 should have a wall thickness of between about 0.010 inches and about 0.050 inches, with a wall thickness of about 0.030 inches preferred.

The proximal end of blunt slide 220 adjacent to slit 222 defines a cut-out portion 224 having a proximally directed angled portion 225 where the apex of the angle points proximally that defines the proximal end of ears 223. Proximally directed angled portion 225 is complementary to dogleg shaped surface 171 at the distal end of proximal lock 170. The proximal end of slot 221 adjacent to slit 222 defines a distally directed angled portion 226 where the apex of the angle points distally that defines the distal ends of ears 223. Distally directed angled portion 226 is complementary to dogleg shaped surface 176 at the proximal end of distal lock 175.

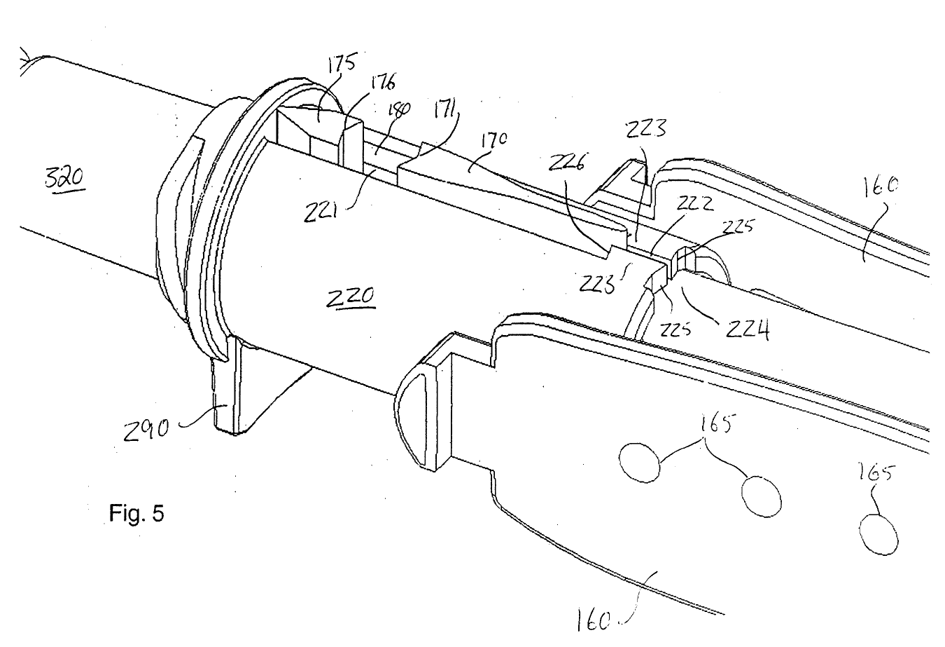

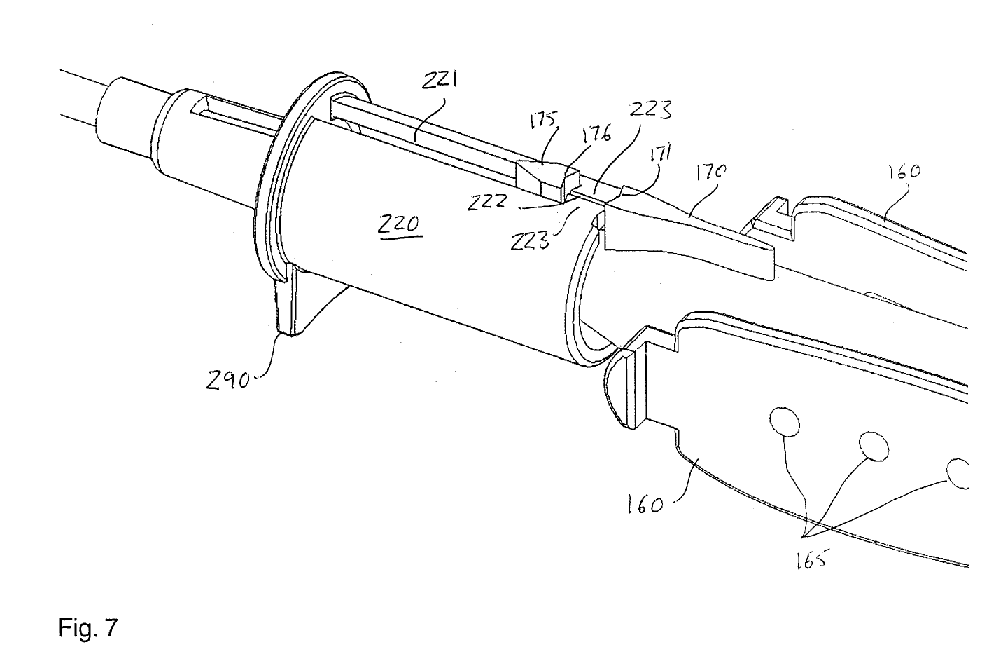

Blunt slide 220 is disposed about needle hub 150 such that proximal lock 170 and distal lock 175 are disposed in slot 221 distal of ears 223 when needle shield 200 is in the proximal position. See FIG. 5. In this position, the proximal portion of proximal lock 170 engages and is disposed in the distal end of slit 222. This facilitates the initiation of movement of needle shield 200 with respect to introducer needle 100 when a clinician moves needle shield 200 to the distal, shielded position. When blunt slide 220 is moved distally with respect to introducer needle 100 the sides of ears 223 defining slit 222 move apart along the tapered sides of proximal lock 170 until ears 223 are adjacent to gap 180. At that point, ears 223 snap back to their original position so that the sides of ears 223 are adjacent to one another and ears 223 are located in gap 180. See FIG. 7. Slot 221 and thus proximal lock 170 and distal lock 175 have a maximum width of between about 0.030 inches and about 0.10 inches, with a maximum width of about 0.060 inches preferred.

When ears 223 are located in gap 180, distally directed angled portion 226 of ears 223 mechanically engages dogleg shaped surface 176 of distal lock 175 to prevent any further relative distal movement of blunt slide 220. The complementary distally directed angled portion 226 of ears 223 and dogleg shaped surface 176 of distal lock 175 increases the force necessary to push ears 223 distally past distal lock 175 so as to be practically impossible. Similarly, when ears 223 are located in gap 180, proximally directed angled portion 225 of ears mechanically engages dogleg shaped surface 171 of proximal lock 170 to prevent any further relative proximal movement of blunt slide 220. The complementary proximally directed angled portion 225 of ears 223 and dogleg shaped surface 171 of proximal lock 170 increases the force necessary to push ears proximally past proximal lock 170 so as to be practically impossible. Thus, blunt slide 220 is locked in position relative to needle hub 150 so needle shield 200 is in the distal, shielded position. This angle for distally directed angled portion 226, dogleg shaped surface 176, proximally directed angled portion 225 and dogleg shaped surface 171 should be between more than 0 degrees and about 60 degrees, with an angle of about 20 degrees preferred.

As can be seen, by optimizing the relationship between the maximum width of slot 221, proximal lock 170 and distal lock 175 with the wall thickness and maximum length of ears 223 as well as the angle of angled portions 225 and 225 and dogleg shaped surfaces 175 and 176, the force needed to activate needle shield 200 can be minimized and the force needed to defeat needle shield 200 can be maximized. Moreover, the preferred throw length of blunt slide 220 can be achieved by optimizing the relationship of the configuration of slot 221, proximal lock 170, proximal lock 175 and ears 223 as described above.

When introducer needle assembly 10 is used as a peripheral IV catheter introducer, catheter assembly 300 is removably disposed about introducer needle assembly 10. Catheter assembly 300 includes a catheter 310 that has a proximal end, a distal end and a catheter hub 320 affixed to the proximal end of catheter 310. Suitable materials for catheter 310 include, but are not limited to, thermoplastic resins such as polyfluoroethylenepropylene (FEP), polytetrafluoroethylene (PTFE), polyurethane and the like. Preferably, catheter 310 is formed from a thermoplastic hydrophilic polyurethane that softens with exposure to physiological conditions present in the patient's body. Suitable materials for catheter hub 320 include, but are not limited to, thermoplastic polymeric resins such as polycarbonate, polystyrene, polypropylene and the like.

When introducer needle assembly 10 is used as a guidewire introducer, catheter assembly 300 can be used as the guidewire introducer or introducer needle assembly 10 can be used alone as the guidewire introducer. However, even where a separate introducer is not needed as a guidewire introducer, it may be desirable to have catheter 310 disposed about external blunt member 210 to minimize venipuncture discomfort to the patient. Without catheter 310, a shoulder between introducer needle 100 and external blunt member 210 will be presented to the patient at the distal end of external blunt member 210. By including catheter 310 that extends past this shoulder, a smoother transition will be presented to the patient making venipuncture easier and less painful.

In order to place introducer needle assembly 10 into a patient's blood vessel, the clinician substantially longitudinally aligns introducer needle 100 with the target blood vessel. The bevel of sharp distal tip 101 should be facing substantially away from the skin surface during venipuncture. The clinician inserts introducer needle 100 at a shallow angle, preferably less than about 35 degrees, into the skin so that sharp distal tip 101 enters the target blood vessel. The clinician, then preferably observes a blood flashback in flashback chamber 155 of needle hub 150.

If introducer needle assembly 10 is to be used as a guidewire introducer, vented plug 190 can be removed from needle hub 150 either before or after successful venipuncture and a guidewire can be inserted through introducer needle assembly 10 into the patient's vasculature. Also, if desired, vented plug 190 can be removed from needle hub 150 so a syringe can be attached to needle hub 150.

If catheter assembly 300 is to be used as a peripheral IV catheter or a guidewire introducer, the clinician advances catheter 310 distally axially along introducer needle 101 into position in the blood vessel. In certain techniques, introducer needle 101 may be partially withdrawn into catheter 310 before catheter 310 is completely advanced into position in the blood vessel. After proper placement of catheter 310 is achieved, the clinician places a finger from her other hand on the patient's skin over the blood vessel approximately over the distal end of catheter 310. By placing her finger on the patient's skin and applying sufficient pressure on the skin, the clinician thereby substantially occludes or at least minimizes blood flow through catheter 310. The clinician then withdraws introducer needle 101 completely from catheter 310 by moving needle hub 150 proximally. Prior to, during or after this withdrawal step, the clinician can push against tab 290 to move blunt slide 220 distally with respect to needle hub 150. This movement causes external blunt member 210 to move distally and shield sharp distal tip 101 of introducer needle 100. After introducer needle 100 and needle shield 200 have been removed from catheter hub 320, the clinician may then attach a fluid delivery device, a PRN, a deadender cap or some other blood monitoring device to catheter hub 320 and commence the planned treatment. Alternatively, catheter assembly 300 can be used to insert a guidewire into a patient. Introducer needle 101 and needle shield 200 may then be disposed of according to the facility's disposal protocol.

Thus, it is seen that an introducer needle assembly with a needle shield is provided that is compact, simple and easy to use and that requires no special features or technique to be operative.

We claim:

1. An introducer needle assembly, comprising:
an introducer needle having a proximal end and a distal end;
a needle hub connected to the proximal end of the introducer needle and including a proximal lock with an angled distal portion and a distal lock with an angled proximal portion wherein the proximal lock is longitudinally displaced from the distal lock to define a gap therebetween;
a needle shield disposed about the introducer needle and having a proximal portion and a distal portion; and
a slide connected to the proximal end of the needle shield and movably disposed about the needle hub wherein the slide defines a slot into which the proximal lock and the distal lock can extend and wherein the slide also defines a pair of ears having an angled proximal end and an angled distal end such that movement of the slide with respect to the needle hub allows the ears to move along the proximally directed tapered portion of the proximal lock and into the gap defined between the proximal lock and the distal lock.

2. The introducer needle assembly of claim 1 wherein the angled distal portion of the proximal lock defines an angle with an apex directed proximally.

3. The introducer needle assembly of claim 2 wherein the angled proximal end of the pair of ears defines an angle with an apex directed proximally.

4. The introducer needle assembly of claim 3 wherein the angle defined by the angled distal portion of the proximal lock is complementary to the angle defined by the angled proximal end of the pair of ears.

5. The introducer needle assembly of claim 3 wherein the angle of the angled distal portion of the proximal lock and the angle of the angled proximal end of the pair of ears is between more than 0 degrees and about 60 degrees.

6. The introducer needle assembly of claim 1 wherein the angled proximal portion of the distal lock defines an angle with an apex directed distally.

7. The introducer needle assembly of claim 6 wherein the angled distal end of the pair of ears defines an angle with an apex directed distally.

8. The introducer needle assembly of claim 7 wherein the angle defined by the angled proximal portion of the distal lock is complementary to the angle defined by the angled distal end of the pair of ears.

9. The introducer needle assembly of claim 8 wherein the angle of the angled proximal portion of the distal lock and the angle of the angled distal end of the pair of ears is between more than 0 degrees and about 60 degrees.

10. An introducer needle assembly, comprising:
an introducer needle having a proximal end and a distal end;
a needle hub connected to the proximal end of the introducer needle and including a proximal lock and a distal lock wherein the proximal lock is longitudinally displaced from the distal lock to define a gap therebetween and the proximal lock and the distal lock have a maximum width of between about 0.030 inches and about 0.10 inches;
a needle shield disposed about the introducer needle and having a proximal portion and a distal portion; and
a slide connected to the proximal end of the needle shield and movably disposed about the needle hub wherein the slide defines a slot into which the proximal lock and the distal lock can extend and wherein the slide also defines a pair of ears such that movement of the slide with respect to the needle hub allows the ears to move along the proximal lock and into the gap defined between the proximal lock and the distal lock.

11. The introducer needle assembly of claim 10 wherein the ears have a thickness of between about 0.010 inches and about 0.050 inches.

12. The introducer needle assembly of claim 10 wherein the ears have a maximum length of between about 0.020 inches and about 0.150 inches.

13. An introducer needle assembly, comprising:
an introducer needle having a proximal end and a distal end;
a needle hub connected to the proximal end of the introducer needle and including a proximal lock and a distal lock wherein the proximal lock is longitudinally displaced from the distal lock to define a gap therebetween;
a needle shield disposed about the introducer needle and having a proximal portion and a distal portion; and
a slide connected to the proximal end of the needle shield and movably disposed about the needle hub wherein the slide defines a slot into which the proximal lock and the distal lock can extend and wherein the slide has a throw length of between about 0.1 inches and about 0.45 inches.
